(19) United States
(12) Patent Application Publication
(10) Pub. No.: US 2024/0282411 A1
(43) Pub. Date: Aug. 22, 2024

(54) Multi-Reference Poly-Conformational Computational Methods for De-Novo Design, Optimization, and Repositioning of Pharmaceutical Compounds

(71) Applicant: Quantori LLC, Cambridge, MA (US)

(72) Inventors: VADIM ALEXANDROV, HOPEWELL JUNCTION, NY (US); YURIY GANKIN, NEWTON, MA (US)

(73) Assignee: Quantori LLC, CAMBRIDGE, MA (US)

(21) Appl. No.: 18/106,479

(22) Filed: Feb. 6, 2023

Related U.S. Application Data
(63) Continuation of application No. PCT/US21/44857, filed on Aug. 6, 2021.
(60) Provisional application No. 63/061,790, filed on Aug. 5, 2020.

(51) Int. Cl. G16C 20/50; G16C 20/70; G16H 70/40

(52) U.S. Cl. CPC G16C 20/50; G16C 20/70; G16H 70/40

(57) ABSTRACT

The invention relates to a fast, alignment-free multi-objective optimization protocol MultiRef3D that maximizes 3D overlap of a query molecule's conformational ensemble with conformational ensembles of multiple reference ligands. Each conformation is treated as an independent entity and characterized by a vector of features (fingerprint) which describes its 3D shape along with the distribution of electrostatic charge across its molecular surface. The method proved to be useful for finding candidate drugs for multi-target disease indications, ligand-based drug design and drug repurposing applications.

### RELATED APPLICATIONS

This application is a continuation of International Application No. PCT/US21/44857, filed Aug. 6, 2021, which claimed the benefit under 35 USC 119(e) of U.S. Provisional Applications No. 63/061,790, filed on Aug. 5, 2020, which are incorporated herein by reference in their entirety.

### BRIEF DESCRIPTION OF THE FIGURES

FIG. 1(a) shows one of the top hits of the multi-target optimization procedure used to screen the Enamine Antiviral virtual library to maximize conformer overlap with all the compounds being tested against SARS-COV-2 in ClinicalTrials.gov.

FIG. 1(b) shows another of the top hits of the multi-target optimization procedure used to screen the Enamine Antiviral virtual library to maximize conformer overlap with all the compounds being tested against SARS-COV-2 in ClinicalTrials.gov.

FIG. 2 shows the original chloroquine as (a) and optimized chloroquine analogs as (b) and (c).

FIG. 3 shows optimization results from Bingo for chloroquine.

FIG. 4 shows the MultiRef3D screening method diagram for the multi-conformer and multi-reference screening procedure. For each test compound, multiple conformers and the corresponding overlapping scores are computed. Later, the overlapping scores are summed into the total score for the selected test compound.

FIG. 5 shows A and B, respectively, the top hits Z1693453146 (Wall=254.11) and ZZ1760146546 (Wall=255.19) from the non-overlapping “antiviral-like” and “Discovery Diversity” libraries.

FIG. 6 shows the active conformers of the Reference compounds (Olaparib, Tadalafil, Ergotamine, and Remdesivir as A, B, C, and D, respectively) aligned with the best-matching conformers of the top hit Z1693453146 (Wall=254.11).

### FIELD OF THE INVENTION

The invention relates to a fast, alignment-free multi-objective optimization protocol MultiRef3D that maximizes 3D overlap of a query molecule's conformational ensemble with conformational ensembles of multiple reference ligands. Each conformation is treated as an independent entity and characterized by a vector of features (fingerprint) which describes its 3D shape along with the distribution of electrostatic charge across its molecular surface. The method proved to be useful for finding candidate drugs for multi-target disease indications, ligand-based drug design and drug repurposing applications.

### Introduction

Conformers as independent molecular entities. Most molecules exist in multiple conformations (shapes). 3D shape of a molecule dictates its biological activity enabling the molecule to fit into binding pockets of proteins. Often distinctly different chemical compounds that have similar shapes (and similar charge distributions along the molecular surface) can bind in a similar way as long as ligand's partial charges are positioned in the binding pocket the same way. Therefore, it is beneficial to compare the shapes and surface distribution charges for query and reference compounds on a conformer-by-conformer basis. If one of the conformers of the query molecule matches one of the conformers (esp. bound-to-target) of the reference molecule, then there is a chance that the reference compound will also exhibit similar binding properties to the same target.

Alignment-free 3D-similarity scoring. OpenEye Scientific Software Inc. pioneered an algorithm ROCS [1] for comparing shapes of molecules by overlaying them in the computer and measuring differences between a query molecule and a target molecule. ROCS is a powerful virtual screening tool which can identify potentially active compounds by shape comparison. ROCS is competitive with, and often superior to, structure-based approaches in virtual screening [2, 3], both in terms of overall performance and consistency [4]. Novel molecular scaffolds have been identified using ROCS against targets often considered very difficult for computational techniques to address [5].

However, overlaying shapes is a computationally intensive process and represents a bottleneck in searching for similar molecules (despite recent, the so-called PAPER implementation of ROCS on GPU [6] and development of FastROCS [7]) for large (>1B) compound libraries. More recent publications describe alternative methods to overlaying, which is accomplished by comparing shape-based descriptors (aka conformer-level 3D fingerprints), e.g. such as ElectroShape implemented in ODDT package [8] based on the algorithms incorporating shape, chirality and electrostatics [9, 10] representing each conformer as a fixed-length vector of real-valued numbers. Another example is a E3FP package which also implements an alignment-invariant 3D representation of molecular conformers as fixed-length binary vector per conformer. These fingerprint-based approaches allow one to calculate similarity between two molecular shapes either as a Tanimoto distance (for binary fingerprints) or Euclidian distance (for real-valued fingerprints) orders of magnitude faster compared to the methods that require actual alignment of the two compared conformers. Calculation of a shape-based fingerprint for each conformer can be a rather involved procedure, but once all conformers for the virtual library are fingerprinted and stored in a database, the similarity search for the query molecule in such database can go rather fast.

### Methods

We generate 3D+surface charge fingerprints for each query molecule conformer and for all conformers of the reference compound. Fingerprints for each of the query molecule conformer are scored using Euclidian distance with respect to all conformers of the reference compound (separately). Fingerprinting individual conformers for the purposes of alignment-free comparisons has become popular in the past couple of years, and we also use these alignment-free approaches for speed (as intended). However, in the fragment-based design applications we identify only the most important part of the fingerprint to screen for sub-structural elements directly involved in a particular target-ligand interaction when multiple (training) ligand examples are available.

Multi-reference Objective Function. The sum of the conformer-to-conformer similarity scores between the query and a reference compound constitutes an objective function W_{c} (for each reference compound c) to maximize. The two variants of the total objective function as the sum of objective functions for each individual reference compound are shown in Eq. 1:

$${W}_{All} = {{\sum }^{C}}_{c = 1} {W}_{c} = {{\sum }^{C}}_{c = 1} {{\sum }^{N}}_{n = 1} {{\sum }^{M}}_{m = 1} {S}_{n , m}^{( c )}$$

$${W}_{max} = {{\sum }^{C}}_{c = 1} {\tilde{W}}_{c} = {{\sum }^{C}}_{c = 1} {{\sum }^{N}}_{n = 1}$$

In Eq 1(a) s_{n,m}^{(c)} is the overlap (e.g. Euclidian distance) of the n's query conformer with the m's conformer for reference compound c, whereas in Eq 1(b) [symbol] is the maximal overlap of the n's query conformer with any of the m conformers of the reference compound c. When one's objective is to identify a novel compound for just one active conformation of one (c=1) reference compound (e.g. a reference ligand co-crystallized with one particular target), then in the above formulas m=1 (all conformers for the query molecule are scored against only one active reference conformer). However, in case if one has multiple reference compounds bound to the same target (or sets of reference compounds bound to multiple targets), the total objective function comes into play. Note that the method is not limited to the structure-based design situations: when several reference compounds were found to be active in a functional assay (and either the target(s) is unknown or the crystal structure of the target is not available)—the formula works just as well. The method becomes especially handy, when there is a great diversity among active reference compounds, whether the target structural information is known or not—the Objective Function will extract and resume all of the relevant parts of the fingerprinted conformer representations responsible for the observed activity.

Multiple reference compounds for the same indication/purpose: examples. The query compound can be evaluated against multiple reference compounds on a conformer-by-conformer basis. In such case, the corresponding similarity scores are summed and constitute the multi-reference conformer-level objective function to maximize (typical use for the ligand-based design).

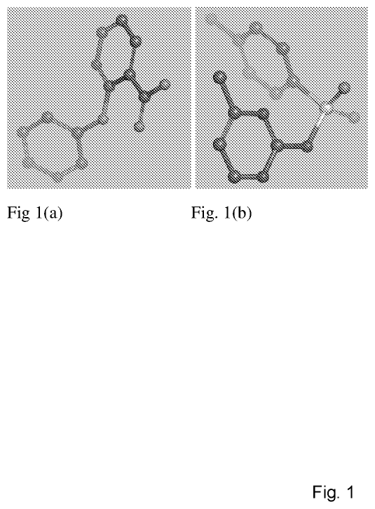

Scoring and optimization (maximization of the total “overlap score”) is shown below on FIG. 1. Column A and B contain query compounds from Enamine REAL database and their overlap scores respectively. The first two compounds (rows 2 and 3) show maximum overall without “gaps”, unlike compound (Virt-cpd-003) whose conformers didn't resemble any of the conformers of Chloroquine, Favipiravir, JQ1 and Apicidine (although a great overlap with conformers from Remdesivir and Haloperidol).

FIG. 1. Query compounds from a virtual library (column A) sorted by their total
overlap score (column B). The numbers in columns C, D, E, etc are respectively the
sums of overlap scores of the conformers for the corresponding reference compounds.

| [symbol] | | B | C | D | E | F | G | H |
|---|---|---|---|---|---|---|---|---|
| 1 | A | Sum | Choloroquine | Remdesivir | Favipavir | JQ1 | Apicidin | Haloperidol |
| 2 | Virt-cpd-001 | 646.99 | 29.2719 | 28.0921 | 59.5148 | 29.8745 | 60.5830 | 61.7833 |
| 3 | Virt-cpd-002 | 609.23 | 37.8701 | 23.4580 | 46.0926 | 39.1227 | 61.0564 | 62.0270 |
| 4 | Virt-cpd-003 | 567.50 | 0 | 62.8056 | 0 | 0 | 0 | 65.0793 |
| 5 | Virt-cpd-004 | 564.74 | 48.7286 | 24.6370 | 36.0694 | 50.5330 | 61.3127 | 61.9991 |
| 6 | Virt-cpd-005 | 564.42 | 2.4828 | 62.4885 | 0.6254 | 2.5460 | 0 | 64.4821 |
| 7 | Virt-cpd-006 | 560.38 | 48.1007 | 25.8045 | 37.2568 | 49.8476 | 60.6062 | 61.3383 |
| 8 | Virt-cpd-007 | 535.61 | 58.2512 | 58.3027 | 59.1439 | 59.3839 | 59.3432 | 59.9429 |
| 9 | Virt-cpd-008 | 534.52 | 8.9032 | 4.6935 | 37.1296 | 7.3091 | 39.1348 | 58.7431 |
| 10 | Virt-cpd-009 | 529.24 | 0 | 55.7474 | 0 | 0 | 0 | 57.5688 |
| 11 | Virt-cpd-010 | 488.97 | 10.5250 | 18.2009 | 31.8874 | 20.0285 | 42.5917 | 52.3405 |
| 12 | Virt-cpd-011 | 478.43 | 0 | 50.3005 | 2.4037 | 0.0000 | 0.6134 | 52.7284 |
| 13 | Virt-cpd-012 | 474.86 | 0 | 0 | 0 | 0 | 0 | 0 |
| 14 | Virt-cpd-013 | 454.42 | 0 | 0 | 33.6453 | 0 | 33.0645 | 45.5773 |
| 15 | Virt-cpd-014 | 449.00 | 0 | 0 | 0 | 0 | 0 | 0 |
| 16 | Virt-cpd-015 | 443.00 | 0 | 0 | 0 | 0 | 0 | 0 |

Special note on the use of non-lowest energy conformations: unlike what majority of computational methods assumed a couple of decade or ago (e.g. in CoMFA method [13]), computational chemists recently started to realize that the bioactive conformation is not necessarily the lowest-energy conformation in the absence of the receptor [14-16]. As long as increase in energy for less favorable conformation is compensated by its binding to the target, i.e. the total ligand-target energy is lower than the sum of the energies for the non-bound target and ligand, the bound state is favored. We heavily use this ligand's ability to use its higher energy conformations depending on the target it attempts to bind. In our approach, however, we compare conformers of the query ligand with conformers from multiple reference compounds whose therapeutic effect of interest is achieved via different mechanisms/binding to different targets, e.g. by inhibiting Main protease (Mpro) and RNA-dependent RNA polymerase (RdRP) [18], while at the same time elevating pH in lysosomes to arrest intracellular proliferation of SARS-COV-2.

Note on applications for structure-based designs: when crystal structure of the target protein is known and the reference ligand is co-crystalized in its active conformation (structure-based design), the query molecules will only be evaluated only with respect to the active reference ligand conformation. Confirmation by direct docking follows for the fingerprint-matched queries.

### Applications of Conformer-Level Multi-Objective Optimization:

Multi-target optimization. We compiled a library of compounds currently undergoing clinical trials from ClinicalTrials.gov (reference compounds). These compounds were identified in PubChem and clustered with respect to their respective Mechanisms of Action (MoA). One hundred conformers for each of the reference molecules were generated at MMFF94 level of theory and each conformer was ODDT-fingerprinted and saved in MongoDB. ODDT implementation [8] of ElectroShape fingerprints was chosen here to demonstrate our approach because these fingerprints are considered to be the state-of-the-art in ligand-based virtual screening experiments [19, 20], and they are not limited to binary values. However, we obtained similar qualitative results when we used other 3D conformer-level binary fingerprint implementation such as E3FP [11].

Our virtual library (query compounds) consisted of Enamine focused virtual sets (Anti-viral, PPI and others) from Enamine database [12]. Molecules from a virtual library were simultaneously evaluated against several anti-viral reference drugs with different mechanism of action (e.g. in SARS-COV-2 case the three major currently pursued MoAs are: ACE2 binding, Mpro and RdRP inhibition). A query molecule for which some of the conformers are similar in shape with conformers for all the reference drugs would receive a higher score.

Optimization from Enamine Anti-Viral-Only Lib+Resulting Molecules:
1. We used the multi-target optimization procedure described above to screen Enamine Anti-viral virtual library to maximize conformer overlap with all the compounds being tested against SARS-COV-2 in ClinicalTrials.gov as of May 15, 2020. Two of the top hits are shown on FIG. 1. One can see immediately from their 3D structures that those are quite flexible molecules due to their either sulphonyl or ether bridge, so they can accommodate different targets.
FIG. 1. Compounds from Enamine virtual anti-viral collection that were found to maximize conformer overlap scores with the currently tested compounds in ClinicalTrials.gov (as of May 15, 2020). Sulphonil bridge (FIG. 1 (b)) is a signature of the classic anti-viral compounds (e.g. well-known drug sulfapyridine), as well as the ether bond. The bridge allows for 3D flexibility for the molecule to change conformation and bind to multiple targets.
2. In-silico optimization of chloroquine:
In-silico synthesis of novel candidate compounds at each optimization iteration: Novel synthetically accessible (synthesizable by an experienced chemist with >90% probability) compound structures that maximize their predicted assay values can be derived in-silico e.g. as implemented in StarDrop's NOVA and BIOSTER modules¹. 1 NOVA can generate new novel molecules by taking a ‘parent’ molecule and creating new generations of related compounds using a collection of over 200 typical ‘medicinal chemistry transformations, whereas BIOSTER brings the collective experience of the chemistry community to derive new active analogues of parent compounds. It contains a unique compilation of over 28,000 transformations including bioisosteric replacements, linker replacements, homologization, and reversible derivatizations (e.g. prodrugs)
Our optimization procedure involves the following steps:
1. Start in-silico synthesis (transformation) of any active compound in the training set by applying chemical transformation rules from chemical reaction databases that ensure tractability (“synthesizability”) of each new “in-silico” compound obtained using these rules (each rule is a documented feasible transformation of a particular compound class or compound substructure).
2. After each transformation, fingerprint all conformers from the obtained novel in-silico compound and calculate the total overlap score with the reference conformers.
3. Based on the value of the total overlap score:
   1) reject novel compounds with lower score
   2) keep transforming compounds with higher score

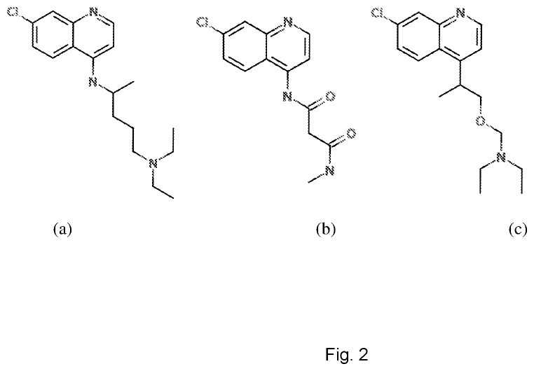

For all datasets that were tested, the above procedure quickly converges to a series of novel compounds with high scores (typically exceeding the highest score in the original set of query compounds).
a) Optimization results based on NOVA in-silico synthesis and selection for higher overall overlap are shown on FIG. 2.

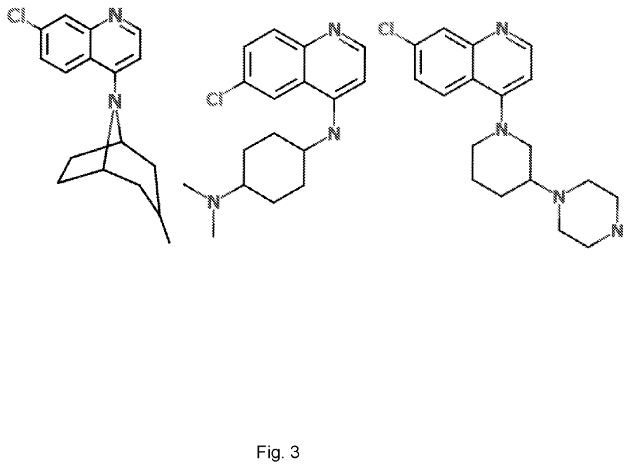

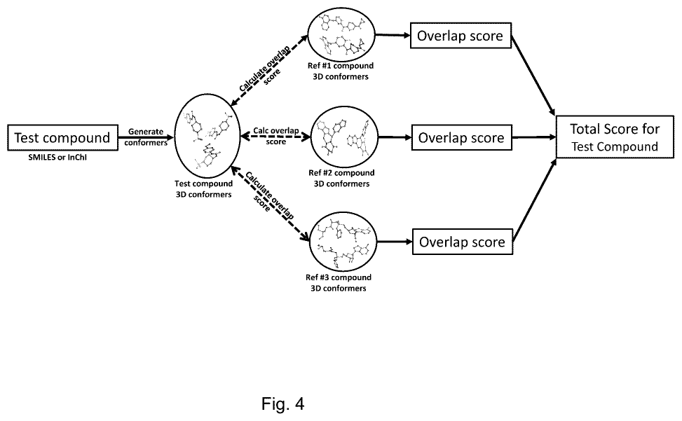

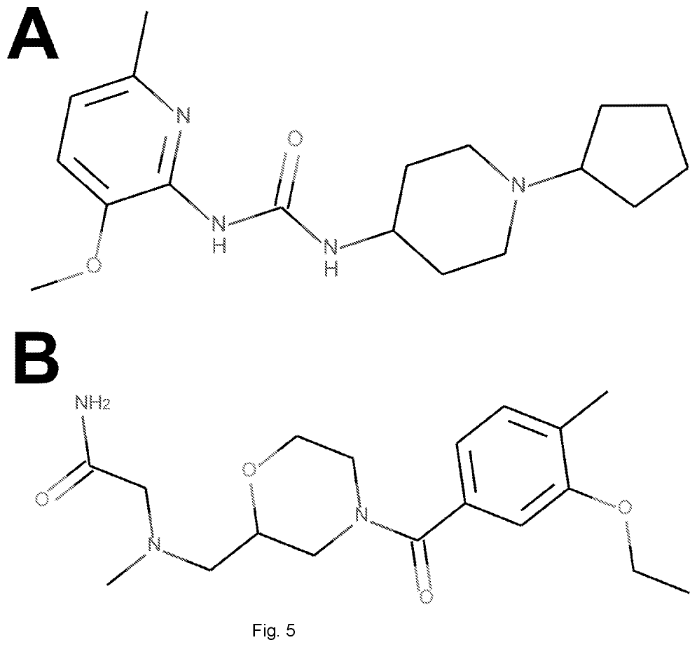

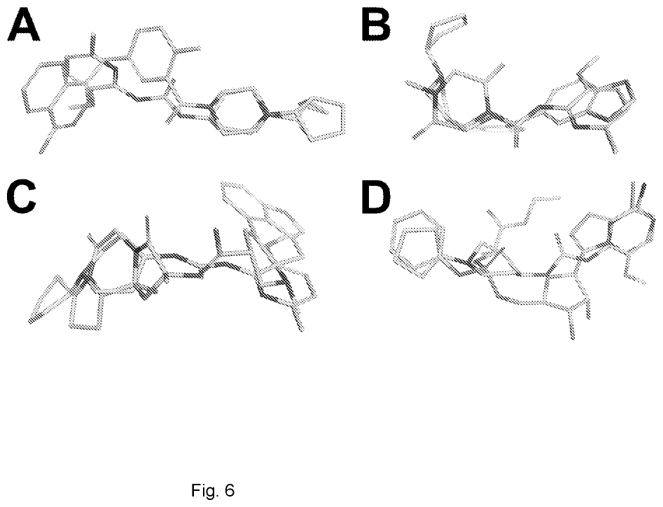

FIG. 2. The original chloroquine is shown on panel (a), optimized chloroquine analogs are shown on panels (b) and (c).
b) Optimization via similarity search. In this exercise, we used EPAM's Bingo similarity search method instead on NOVA in-silico synthesis to “modify” compounds on each iteration. We chose to fingerprint the entire REAL Enamine database of >1.2 B compounds to ensure that the sampled chemistry space is properly covered. The procedure was as follows: on each iteration, fetch analogs from REAL via Bingo for the current set of compounds (with cloroquine being the only compound on the 1^{st} iteration) and select similarity hits with even higher scores for the next iteration. Then on the next iteration, for each higher-scored similarity hit fetch similar compounds again, score them and repeat selection/fetch/score procedure until the score doesn't improve any more. The optimization results from this procedure are shown on FIG. 3.

FIG. 3. Optimization results from Bingo for choloroquine (compare to the original chloroquine structure shown on FIG. 2 (a))

Application for drug-repurposing: depending on what is known about the indication or marketed drug of interest (targets, MoAs, other existing drugs for the same indication) any of the above scenarios and methods (or combination thereof) can be used to find other non-obvious molecules whose shape and surface electrostatic charge is similar to that of the marketed drug and/or the cumulative such similarity to conformers of the drugs used to treat this disease indication.

### Conclusion

We showed usefulness of the multi-reference optimization approach in various in-silico drug discovery settings, both structure-based and ligand-based designs. This work promotes thinking about a molecule as an ensemble of flexible conformers that would choose the best possible conformation for each presented target-binding opportunity.

### Abstract

In this work a novel computational multi-reference poly-conformational algorithm is presented for design, optimization, and repositioning of pharmaceutical compounds. The algorithm searches for candidates by comparing similarities between conformers of the same compound and identifies target compounds whose conformers are simultaneously “close” to the conformers for each of the compounds in a reference set. The reference compounds can have very different MoAs, which directly and simultaneously shapes the properties of the target candidate compounds.

The algorithm functionality has been validated in silico by scoring ChEMBL drugs against FDA-approved reference compounds which either had the highest predicted binding affinity to our chosen SARS-COV-2 targets or confirmed to be inhibiting such targets in-vivo. All our top scoring ChEMBL compounds also turned out to be either high-affinity ligands to the chosen targets (as confirmed separately in other studies) or showing significant efficacy in-vivo against those selected targets.

search for new compounds within two virtual libraries from the Enamine database. The library's virtual compounds have been compared to the same set of reference drugs that we used for validation: Olaparib, Tadalafil, Ergotamine and Remdesivir. The large reference set of four potential SARS-COV-2 compounds have been selected, since no drug has been identified to be 100% effective against the virus so far, possibly because each candidate drug was targeting only one particular MoA. The goal here was to introduce methodology for identifying potential candidate(s) that cover multiple MoA-s presented within a set of reference compounds.

### Keywords

conformers, multi-reference, poly-conformational, in silico, ligand-based, structure-based, SARS-COV-2, COVID-19, fingerprints, cheminformatics, similarity, virtual library, computational framework, validation.

### Introduction

Conformers as independent molecular entities. In real life, most compound molecules exist in multiple conformations (shapes) based on the surrounding environmental conditions. In particular, each 3D shape of a molecule dictates its biological activity and enables the molecule to fit into the binding pockets of proteins. Often, distinctly different chemical compounds that have similar shapes (and similar charge distributions along the molecular surface) have a potential to bind as long as the ligand's partial charges are positioned in the binding pocket the same way (i.e., form the same hydrogen bonds). Therefore, it is beneficial to compare the shapes and surface distribution charges for target query and reference compounds on a conformer-by-conformer basis. If one of the conformers of the query molecule matches one of the conformers (especially bound-to-target) of the reference molecule, then there is a chance that the reference compound will also exhibit similar binding properties to the same target.

Alignment-free 3D-similarity scoring. OpenEye Scientific Software Inc. pioneered an algorithm and the corresponding tool ROCS¹³ for comparing shapes of molecules by overlaying and measuring their molecular structures in silico and comparing differences between a query and target molecule. ROCS identifies potentially active compounds by comparing their shapes. Moreover, the ROCS tool is competitive and often superior to structure-based approaches in virtual screening^{14,15} both in terms of overall performance and consistency¹⁶. As a result, novel molecular scaffolds have been identified by using ROCS against various targets which have been considered very difficult to address computationally¹⁷.

Challenges with overlaying. The process of molecular shapes overlaying remains computationally intensive and often is a bottleneck in the search process for similar molecules. This remains despite the recent so-called PAPER implementation of ROCS on GPU¹⁸ and the development of FastROCS¹⁹ for large (>1B) compound libraries. Recently, alternative methods for overlaying have been introduced as a substitute for the ROCS approach. The alternative overlaying is performed by comparing shape-based descriptors (a.k.a conformer-level 3D fingerprints). An example of such an approach is ElectroShape implemented in the ODDT package²⁰ and is based on the algorithm that incorporates shape, chirality, and electrostatics^{21,22}, and represents each conformer via a fixed-length vector of real-valued numbers. Similarly the E3FP package²³ also utilizes an alignment-invariant 3D representation of molecular conformers as a fixed-length binary vector for each conformer. These fingerprint-based approaches allow to calculate the similarity between two molecular shapes either as a Tanimoto distance (for binary fingerprints) or Euclidean distance (for real-valued fingerprints) computations. Such computations are orders of magnitude faster in comparison to alternative methods that require the actual alignment of the two compared conformers. Even though the calculation of a shape-based fingerprint for each conformer can be a rather computationally involved procedure, as soon as all conformers for the virtual library are fingerprinted and stored in a database, the similarity search for the query molecule in such a database is computationally quick. Therefore the computationally efficient method proposed here is expected to be very useful for finding candidate drugs for multi-target disease indications, ligand-based drug design, and drug repurposing applications.

Method applications for SARS-COV-2 treatment compounds. The set of SARS-COV-2 treatment compounds have been used for both method validation since, there are compounds that have been confirmed to be effective²⁴ and for the search for new potential compounds based on the existing known set since no drug has been identified to be 100% effective against the virus, possibly because each candidate drug was targeting only one particular MoA²⁵. The SARS-COV-2 virus has been selected for method illustration because of the importance of the subject. The virus was introduced into the human population in the Chinese city of Wuhan in the Province of Hubei in December of 2019¹⁻⁴. Since then the epidemic of SARS-COV-2 has rapidly spread Worldwide. The World Health Organization (WHO) has officially declared the SARS-COV-2 pandemic in March 2020 just three months after its emergence⁵. The novel coronavirus received an official name SARS-COV-2 and the virus pandemic was called COVID-19⁶. The formal evaluation and comparison of SARS-COV-2 drugs can be performed by studying the compound properties by treating patients and performing clinical trials^{7-10,12} or by studying the properties of the corresponding compounds in silico^{9,11} which is done in this work. For method validation we used public ChEMBL (version 28) database²⁶ to screen compounds against the most important viral targets, namely 3C-like protease (3CLpro, aka Main protease or Mpro), papain-like protease (PLpro) and RNA-dependent RNA polymerase (RdRp). These targets play a major role in the replication/transcription and host cell recognition and therefore, are vital for the viral reproduction and spread of infection. Because our method doesn't directly use target information but rather analyzes 3D shapes for a compound that was already predicted or experimentally found to be effective against a particular target (we call it a reference compound), one has to choose one (or more) such compound(s) as a reference for each target. The focus for each of the above SARS-COV-2 targets (3CLpro, PLpro and RdRp) was on the reference compounds with the highest binding affinities from the recent in silico multi-target repurposing study²⁴.

For the new compound search (virtual library screening) we used the same set of reference compounds as we used for the method validation.

### Methods

Representative conformer space and conformer-by-conformer comparison. The proposed computational algorithm extends the currently available methods²⁰⁻²³ and introduces additional search flexibility via the use of the compound conformers. The proposal is to compare multiple possible shapes, adopted via varying environmental conditions, of the same molecule (i.e., conformers) rather than just a single shape that was used before. In particular, the suggested approach is based on the matching of ligand-ligand fingerprints without explicitly using target structure information unlike docking and molecular dynamics approaches that simulate physical binding of a ligand to the target. The supporting theory behind the method is based on the decision to treat conformers, which might have different binding characteristics and properties, as independent entities. In such an approach each conformer has the corresponding independent alignment-free 3D-similarity scoring using the known multi-references. All conformers were generated using the ETKDG algorithm implemented in RDkit²⁷. Benchmarking studies have found ETKDG to be the best-performing freely available conformer generator up-to-date^{28,29} providing diverse and chemically-meaningful conformers reproducing crystal conformations. Unlike what the majority of computational methods had assumed a couple of decades or so ago (e.g. in the CoMFA method⁵⁸), recent research indicates that the bioactive conformation is not necessarily the lowest-energy conformation in the presence of the receptor⁵⁹⁻⁶¹. In particular, as long as an increase in energy for less favorable conformation is compensated by its binding to the target, i.e. the total ligand-target energy is lower than the sum of the energies for the non-bound target and ligand, the bound state is favored. The proposed method emphasizes and relies on this ligand's ability to use its potentially higher energy conformations depending on the target it attempts to bind. Note, however, that when a sufficiently large number of conformers is requested, ETKDG algorithm generates more conformers with lower energy than with higher energy^{27,28}, therefore when averaged over all conformers (and we generate 100 conformers per molecule), conformers with the lower energy will contribute more to the total overlap.

Actually, one of the things that distinguishes ligand-based 3D virtual screening methods from 2D methods is that one has to start worrying about how many conformers to include in the reference set. If the molecule is flexible, it can assume many shapes and pharmacophores. How to deal with this is one of the fundamental questions in ligand-based virtual screening (LBVS).

In a recent paper by Schrödinger team³⁰ Cappel et al. performed comprehensive benchmark analysis and found that the number of conformers needed for 3D LBVS is actually relatively low: 100 or less to achieve good performance. Thus, we used ETKDG to generate 100 conformers per molecule in this work.

The authors have called the approach MultiRef3D to emphasize that it is a fast, alignment-free multi-objective optimization protocol that maximizes the 3D overlap of a query molecule's conformational ensemble with conformational ensembles of multiple reference ligands. The diagram of the proposed method is summarized in FIG. 1. The formal details of the approach are discussed further.

FIG. 1. MultiRef3D screening method diagram for multi-conformer and multi-reference screening procedure. For each test compound multiple conformers and the corresponding overlapping scores are computed. Later, the overlapping scores are summed into the total score for the selected test compound.

Efficiency and a conformer scoring. In the algorithm, each conformation is treated as an independent entity and is characterized by a vector of features (fingerprint) which describes its 3D shape along with the distribution of electrostatic charge (both denoted further as electroshape) across its molecular surface. In this work we used 15-dimensional USRCAT fingerprints³¹ which distil molecular shape into a rotation-invariant descriptor vector made up of 15 real numbers describing distance distribution among atoms, atomic partial charges and atom types. USRCAT fingerprints were shown to significantly outperform just shape-based fingerprints in recent benchmark tests^{31,32}. Since USRCAT fingerprints reflect both relative 3D positions for all atom types and molecular surface charges for each query molecule conformer as well as for all conformers of the reference compound they are very well-suited for alignment-free fast computation of conformer similarity. Each conformer is coded within the algorithm by a single fingerprint represented as a fixed-length vector of numbers which ensures computational efficiency. These fingerprints for each of the query and reference molecule conformers are individually scored by Euclidean distance serving as a similarity measure between two conformers. The Euclidean distance can be viewed as an extension of the Tanimoto similarity measure for non-binary fingerprints. The fingerprinting of individual conformers for alignment-free comparisons became popular in the past couple of years^{23,33-35} so the proposed method is built on those.

Objective Function Optimization. The sum of the conformer-to-conformer similarity scores between the query and a reference compound are compared via an objective similarity function W_{c} for each reference compound c. The goal is to maximize the sum of those individual objective similarity functions across all reference compounds of interest c=1, 2, . . . , C where c is a summation index for the desired set of reference compounds:

$${W}_{All} = {{\sum }^{C}}_{c = 1} {W}_{c} = {{\sum }^{C}}_{c = 1} {{\sum }^{Q}}_{q = 1} {{\sum }^{R}}_{r = 1} {S}_{q , r .}^{( c )} \qquad ( 1 )$$

In formula (1) the summand S_{q,r}^{(c)} is the similarity (overlap) of the query conformer q (q=1, 2, . . . , Q) with the conformer r (r=1, 2, . . . , R) for each reference compound c (c=1, 2, . . . , C). For the real-valued fingerprints, the similarity summand between the pair of conformers of interest indexed by query index q and reference index r for compound c is calculated as:

$${S}_{q , r}^{( c )} = 1 - ( 1 / N ) \sqrt{{{\sum }^{N}}_{n = 1} {( {x}_{q , n}^{( c )} - {x}_{r , n}^{( c )} )}^{2}} \qquad ( 2 )$$

where x_{q,n}^{(c)} and x_{r,n}^{(c)} are the corresponding normalized fingerprint vector coordinates for n=1, 2, . . . , N. The length (the number of coordinates) of the fingerprint N is determined based on the problem-specific target-ligand interaction characteristics. Since the fingerprint coordinates x_{q,n}^{(c)} and x_{r,n}^{(c)} are normalized (i.e. have values between 0 and 1 for each coordinate n) the resulting q,n r,n overlap s_{q,r}^{(c)} is maximized with the value equal to 1 when the fingerprints of both conformers are identical and can take the smallest value equal to 0 when all the fingerprint coordinates have a difference equal to 1 i.e. as different as possible at the normalized scale.

When the objective is to identify a novel compound for just a single active conformation (r=1) of one (c=1) reference compound (e.g. a reference ligand co-crystallized with one particular target) then all conformers for the query molecule are scored against only one active reference conformer. However, in the case when multiple reference compounds are bound to the same target (or sets of reference compounds bound to multiple targets), the total objective function comes into play. It is important to point out that the proposed method is not limited to the structure-based design situations: when several reference compounds are found to be active in a functional assay (and either the target(s) is unknown or the crystal structure of the target is not available)—the formula works just as well (as long as the ligand structure is known). The method becomes especially handy, when there is a great diversity among active reference compounds, whether the target structural information is known or not—the objective function will extract and sum up the similarities for all of the relevant parts of the fingerprinted conformer representations responsible for the observed activity.

The query compound can be evaluated against multiple reference compounds on a conformer-by-conformer basis. In such a case, the corresponding similarity scores are summed and constitute the multi-reference conformer-level objective function to maximize. This can be readily used in a typical ligand-based design setting. However, instead of just searching for a shape analog of one of the conformers of a reference compound, in the case of multiple references, the algorithm performs a search for such a compound in the virtual library whose conformers have overlapped with conformers of each of those reference compounds. The latter will increase the chances that the selected virtual compound binds the same way to the corresponding targets of each of the references (i.e. the selected compound is capable of forming conformations that resemble active conformations responsible for the MoA of each of the references).

Method validation for known targets. To validate the proposed methodology for the multi-target-specific conformer similarity the three following targets have been used: 3CLpro (Mpro), PLpro, and RdRp. The spike protein has not been included as the validation target since the pharmacological activity may not be correlated directly with the binding affinity to the interfacial site²⁴. ChEMBL (version 28) public database²⁶ has been chosen as the universe for screening. The selected ChEMBL compounds were already marketed drugs for which at least one target is known. The corresponding ChEMBL extraction query is provided in the manuscript Supplement. The screened set had a total of 2,604 compounds. The corresponding reference compounds for validation were selected from the recent multi-target in silico repurposing study²⁴ based on the highest binding affinities for each of the SARS-COV-2 three targets 3CLpro, PLpro and RdRp.

Compounds search based on reference compounds' conformers. One hundred conformers for each of the reference molecules were generated at the MMFF94 level of theory⁴⁰ and each conformer was ODDT-fingerprinted²⁰ and saved in the MongoDB database⁴¹. The ODDT implementation²⁰ of ElectroShape fingerprints²² has been selected to demonstrate the proposed approach because these fingerprints are considered to be state-of-the-art in ligand-based virtual screening experiments^{32,42}, and they are not limited to binary values.

Virtual libraries for screening. Virtual libraries (query compounds) for screening consisted of Enamine⁴¹ focused “antiviral-like” set (3,995 compounds) and diverse Discovery Diversity Set (10,559 compounds)⁴⁴. Molecules from each virtual library were simultaneously evaluated against several reference drugs with different MoA (3CLpro, PLpro and RdRp inhibition). A query molecule for which some of its conformers are similar in shape with conformers for all the reference drugs would receive a higher score. In this approach, multiple virtual compounds can be identified to have a good conformer overlap with conformers of the reference drugs.

### Results

Method Validation for SARS-COV-2 Compounds. The highest affinity binder Olaparib (−9.2 kcal/mol) has been selected as a reference compound for 3CLpro, Tadalafil (−9.2 kcal/mol) for PLpro and Lumacaftor (−9.9 kcal/mol) for RdRp. However, when multi-target scoring against these three references has been performed, the top ten scoring compounds from ChEMBL had no conformers similar in 3D shape (Euclidean distance<0.5) to Lumacaftor conformers. Therefore, the Lumacaftor reference has been replaced with the next best in silico RdRp binder Ergotamine²⁴ (−9.4 kcal/mol binding affinity to RdRp). The resulted scores produced by the proposed method are summarized in Tab. 1:

TABLE 1
Top ten scoring compounds showing simultaneous conformer similarity
with the reference compounds Olaparib, Tadalafil, and Ergotamine.

| Compound ID | Compound Name | TotalScore | Olaparib | Tadalafil | Ergotamine |
|---|---|---|---|---|---|
| CHEMBL779 | Tadalafil | 228.46 | 70.70 | 100.00 | 57.76 |
| CHEMBL1737 | Sildenafil citrate | 225.15 | 81.30 | 58.34 | 85.50 |
| CHEMBL521686 | Olaparib | 223.08 | 100.00 | 57.61 | 65.48 |
| CHEMBL105442 | Ci-1040 | 220.40 | 80.68 | 79.16 | 60.56 |
| CHEMBL129857 | As-602868 | 220.16 | 78.27 | 74.50 | 67.39 |
| CHEMBL2037511 | Epelsiban | 219.86 | 81.58 | 70.28 | 68.01 |
| CHEMBL565612 | Sotrastaurin | 219.13 | 79.93 | 69.36 | 69.83 |
| CHEMBL1516474 | Tegaserod maleate | 217.83 | 80.22 | 76.56 | 61.05 |
| CHEMBL1236682 | Refametinib | 217.78 | 76.01 | 81.57 | 60.20 |
| CHEMBL1923502 | Ulimorelin | 217.56 | 76.29 | 74.79 | 66.47 |
| | hydrochloride | | | | |

Both Olaparib and Tadalafil had the highest scores which confirmed the previous finding²⁴ that these compounds are simultaneously good binders for both 3CLpro and PLpro. Our method has also picked up Sildenafil (more commonly known under the brand name Viagra) which just like Tadalafil (aka Cialis) is also known as a classical PDE5A inhibitor. Although those compounds are predominantly used in the treatment of male erectile dysfunction and pulmonary hypertension, it was shown 45 that in the presence of SARS-COV-2 infection, PDE5 inhibitors prevent thromboembolism caused by inflammatory processes in COVID-19 patients via NO/cGMP pathway and are potent inhibitors of 3CLpro⁴⁶.

Ci-1040 and Refametinib are the other two hits from Tab. 1 and are potent MEK inhibitors with high 3D shape similarity to both Olaparib and Tadalafil. MEK inhibitors, including Olaparib⁴⁷ were recently demonstrated to reduce cellular expression of ACE2 while stimulating NK-mediated cytotoxicity and attenuating inflammatory cytokines during the severe stage of SARS-COV-2 infection⁴⁸. Ci-1040 was also previously shown to display a broad anti-influenza virus activity in vitro and to provide a prolonged treatment window compared to the standard of care in vivo, specifically in lung cells⁴⁹.

The other hit from Tab. 1 is Sotrastaurin which is a PKC inhibitor and has been experimentally shown to inhibit SARS-COV-2 replication in vivo⁵⁰ and has been found to be among the best 3CLpro binders during in silico ZINC database screening study⁵¹ Yet another notable hit among the top ten selected compounds in Tab. 1 is As-602868: a potent IKK2 inhibitor. This class of compounds is currently preclinically tested for NF-kB mediated cytokine storm attenuation in severe COVID-19 patients⁵².

The other top hit, Epelsiban, was originally developed as an oxytocin receptor agonist. However, it has been recently shown⁵⁴ that oxytocin plays a major role in activation of NF-kB-mediated pathways. Interestingly, recent research has revealed 50 that Remdesivir (in addition to being a potent RdRp inhibitor) is also reducing viral replication via NF-kB pathway. Therefore, this hit serves as an example of non-obvious 3D-shape-based drug repurposing idea generation linked to the relevant yet non-primary SARS-COV-2 inhibiting mechanisms of reference compounds.

Thus in our second validation experiment the RdRp reference compound Ergotamine has been replaced with Remdesivir which, as we already mentioned, is a well-established RdRp inhibitor and cytokine storm attenuator that works via NF-kB pathway. The resulted scores produced in the second scoring setup are summarized in Tab. 2.

TABLE 2
Top ten scoring compounds showing simultaneous conformer similarity
with the reference compounds Olaparib, Tadalafil, and Remdesivir.

| Compound ID | Compound Name | TotalScore | Olaparib | Tadalafil | Remdesivir |
|---|---|---|---|---|---|
| CHEMBL1694 | Benazepril | 180.82 | 66.67 | 64.26 | 49.89 |
| | hydrochloride | | | | |
| CHEMBL515606 | Cilazapril | 180.61 | 64.56 | 61.56 | 54.50 |
| CHEMBL495727 | At-9283 | 179.03 | 68.17 | 56.15 | 54.71 |
| CHEMBL2107495 | Temafloxacin | 178.94 | 67.15 | 55.78 | 56.01 |
| | hydrochloride | | | | |
| CHEMBL1200779 | Trovafloxacin | 178.60 | 66.24 | 54.02 | 58.35 |
| | mesylate | | | | |
| CHEMBL340978 | Benoxaprofen | 178.27 | 68.56 | 56.54 | 53.16 |
| CHEMBL8 | Ciprofloxacin | 177.05 | 63.21 | 57.19 | 56.65 |
| CHEMBL1200831 | Spirapril | 177.00 | 65.28 | 60.24 | 51.47 |
| | hydrochloride | | | | |
| CHEMBL1201011 | Quinapril | 176.84 | 66.32 | 60.40 | 50.13 |
| | hydrochloride | | | | |
| CHEMBL1168 | Ramipril | 176.54 | 65.32 | 63.26 | 47.96 |

For Olaparib, Tadalafil, and Remdesivir reference compounds half of the top ten hits (Benoxaprofen, Ciprofloxacin, Spirapril hydrochloride, Quinapril hydrochloride and Ramipril) turned out to be ACE inhibitors and coagulation modifiers acting via NF-kB related pathways^{55,56}! In addition, all of them turned out to be also good binders of 3CLpro⁵⁷.

The other hits were Temafloxacin and Trovafloxacin, predicted to be potent 3CLpro ligands⁵⁵ and experimentally shown to inhibit virus replication^{56,57}, and anti-inflammatory drugs Benoxaprofen and Ciproflaxin predicted to target 3CLpro^{58,59} as well.

Concluding the list is an interesting multi-target Aurora/JAK inhibitor hit: compound At-9283. JAK inhibitors, in general, have promising therapeutic potential for SARS-COV-2 treatment with their dual anti-inflammatory and anti-viral effects⁶⁰. At-9283, however, has also been recently identified to reverse SARS-COV-2 transcriptomic signature⁶¹ and due to its matching tipiracil's 3D pharmacophore scaffold also inhibits SARS-COV-2 Nsp15 endoribonuclease^{62,63} and targets 3CLpro as well^{64,65}.

Virtual library screening for multi-target SARS-COV-2 compounds. The results from the focused (“antiviral-like”) and diverse (“Discovery Diversity Set”) are summarized in Tab 1 and 2 respectively. The algorithm visual summary is displayed in FIG. 1 for the W_{All} objective function. Tables 3 and 4 summarize the direct application results of the Enamine⁴¹ focused “antiviral-like” and “Diverse Discovery Set” virtual library screening. The first two columns of the Tables contain query compound IDs and their computed overlap scores. The rows are sorted according to the total sum overlap score displayed in the second column.

TABLE 3
The top scoring compounds from the Enamine “antiviral-like” virtual
library (the first column) are sorted by their total overlap score W_{All} (the
second column). The values in the other columns correspond to the sums of
the overlap scores of the conformers for the corresponding reference compounds.

| Compound ID | Wall | Olaparib | Tadalafil | Ergotamine | Remdesivir |
|---|---|---|---|---|---|
| Z1693453146 | 254.11 | 71.68 | 56.30 | 76.76 | 49.38 |
| Z434669842 | 248.84 | 66.18 | 55.33 | 69.92 | 57.41 |
| Z1381427631 | 248.57 | 66.63 | 52.86 | 72.84 | 56.24 |
| Z1381425049 | 247.97 | 66.09 | 53.03 | 71.98 | 56.88 |
| Z1313285936 | 246.97 | 67.70 | 55.51 | 72.23 | 51.54 |
| Z826278840 | 246.37 | 65.61 | 56.67 | 68.79 | 55.30 |
| Z94559538 | 245.69 | 70.41 | 55.41 | 65.24 | 54.64 |
| Z435640438 | 245.21 | 63.85 | 54.62 | 72.45 | 54.29 |
| Z435642248 | 245.13 | 64.04 | 55.31 | 71.85 | 53.93 |
| Z827564114 | 244.89 | 64.93 | 57.02 | 70.19 | 52.75 |

TABLE 4
The top scoring query compounds from the Enamine a “Diverse Discovery Set”
virtual library (the first column) are sorted by their total overlap score W_{All} (the
second column). The values in the other columns correspond to the sums of the
overlap scores of the conformers for the corresponding reference compounds.

| Compound ID | Wall | Olaparib | Tadalafil | Ergotamine | Remdesivir |
|---|---|---|---|---|---|
| Z1760146546 | 255.19 | 74.41 | 60.57 | 74.94 | 45.27 |
| Z3077896041 | 254.26 | 67.67 | 57.38 | 75.53 | 53.67 |
| Z2911083836 | 253.23 | 72.49 | 58.63 | 75.85 | 46.27 |
| Z2446617864 | 252.49 | 73.18 | 59.52 | 75.06 | 44.72 |
| Z1139281415 | 252.30 | 69.01 | 57.39 | 75.88 | 50.02 |
| Z1354703942 | 251.79 | 77.22 | 61.63 | 80.63 | 32.32 |
| Z2256366543 | 251.27 | 66.54 | 54.27 | 74.18 | 56.28 |
| Z1139281396 | 250.99 | 68.40 | 57.90 | 74.43 | 50.27 |
| Z1139280685 | 250.51 | 68.34 | 57.88 | 74.50 | 49.79 |
| Z2959367287 | 250.32 | 69.54 | 55.40 | 72.80 | 52.58 |

For the visual illustration of the algorithm results two compounds with the highest scores from Tab. 3 and 4 have been presented in FIG. 2, panels A and B respectively. It is worth noting that these compounds are quite flexible molecules due to their amidebridge around which the ring substructures can rotate, which ensures the ability of those molecules to accommodate different targets.

FIG. 2. The compounds presented in panels A and B are the top hits Z1693453146 (Wall=254.11) and ZZ1760146546 (Wall=255.19) from the non-overlapping “antiviral-like” and “Discovery Diversity” libraries respectively. One can immediately observe, however, that the compounds share a lot of similarity, in particular overall shape and amide bridge connecting heterocycles. The bridge allows for 3D flexibility for the molecule to change conformation and bind to multiple targets.

FIG. 3 demonstrates how the best-matching conformers of the top hit Z1693453146 spatially align with the active conformation for each reference drug. One can observe that the majority of hydrogen donors and acceptors from the top hit conformer and reference conformers are aligned very well mimicking the interaction patterns with each target. At least partial spatial alignment of atom types is expected from the top hit conformers since atom types as well as their relative 3D positions is the essence of the USRCAT fingerprints³¹.

FIG. 3. Active conformers of the Reference compounds (Olaparib, Tadalafil, Ergotamine and Remdesivir on panels A, B, C and D respectively) aligned with the best matching conformers of the top hit Z1693453146 (Wall=254.11). Carbon-carbon bonds for the reference compounds and the tTop hHit are shown in green and cyan respectively (C—N and C—O bonds are conventionally shown in blue and red).

### Discussion

Computation efficiency and availability of the method. The proposed method does not rely on laborious docking and molecular dynamics setup, especially in the multi-target case, where target preparation and choice of method i.e. direct docking to a fixed-coordinate target or Molecular Dynamics-based ensemble energy minimization are of utmost importance and require deep expertise. Fingerprint comparison is orders of magnitude faster and simpler (only requires simple structural information in the form of either isomeric SMILES or InChI). The entire setup is presented in our Supplement that can be universally used for any multi-target screening and optimization whenever reference compounds for each of the targets are available. Naturally, further hit refinement (ADMETox, PK/PD, etc) is necessary if the screened universe is not limited to the drugs with the well-known safety profiles.

Application for drug-repurposing. Depending on what is known about the indication or marketed drug of interest (targets, MoAs, other existing drugs for the same indication) the proposed methods (or a combination thereof) can be used to find other non-obvious molecules whose shape and the surface electrostatic charge is similar to that of the marketed drug. The methods can also be used to search for the cumulative similarity to conformers of the multiple drugs used to treat this disease indication.

n the proposed approach multiple conformers of the query ligand have been compared with conformers from multiple reference compounds whose therapeutic effect of interest is achieved via different mechanisms of bindings to different targets, e.g. by inhibiting major proteases 3CLpro and PLpro⁶³ and RNA-dependent RNA polymerase (RdRp)^{6465,66}. An “ideal drug” would contain conformers that resemble (as many as possible) conformers of all of the reference drugs, thus increasing chances that the drug inhibits SARS-COV-2 via multi-MoA routes and is more effective than each individual reference drug.

Note on applications for structure-based designs. When the crystal structure of the target protein is known and the reference ligand is co-crystallized in its active conformation (structure-based design), we can use this information about the reference compound and evaluate the query molecules against only one, the active (co-crystallized) reference ligand conformation (r=r_{active}) in formulas (1) and (2). Confirmation by direct docking for the fingerprint-matched queries can be used to confirm the match.

Our methodology emphasizes pursuit of candidate compounds that achieve therapeutic effect (e.g. stops SARS-COV-2 proliferation) by multiple MoA routes. A successful candidate compound would contain conformers targeting the two major proteases 3CLpro and PLpro RdRpall at the same time by increasing chances that the compound would protect against SARS-COV-2 much more effectively. Naturally, all successful candidates would need to be further screened and filtered for proper ADME-Tox and other drug-likeness properties. Binding to anti-targets, e.g. hERG, can be explicitly incorporated to this methodology by adding the corresponding terms (similarities to known hERG-binding ligands) to the overlap sum with a negative sign. Even though many computational methods exist to evaluate hERG in particular as well as other common tox liabilities, when an anti-target is very specific and less commonly known as “pure tox target” (e.g. undesired binding to D2 receptor for many modern CNS drugs), the explicit inclusion of similarity score to such anti-target with a negative sign can greatly streamline the overall drug optimization process.

### Conclusion

We have demonstrated and validated the usefulness of the multi-reference computationally efficient optimization approach in drug discovery screening and repurposing scenarios. The method represents each molecule as an ensemble of flexible conformers that would choose the best possible conformation for each presented target-binding opportunity. Application of this approach to SARS-COV-2 produced several antiviral drug candidates that are designed to protect against SARS-COV-2 by multiple mechanisms simultaneously.

### LIST OF ABBREVIATIONS

ADME-Tox—Absorption, Distribution, Metabolism, Excretion and Toxicity
GPU—Graphics processing unit
CNS—Central nervous system
CoMIFA—Comparative molecular field analysis
COVID-19—Coronavirus Disease of 2019
CPU—Central processing unit
hERG—Human Ether-a-go-go-related Gene
MoA(s)—Mechanism of Action(s)
ODDT—Open Drug Discovery Toolkit
RNA—Ribonucleic acid
ROCS—Rapid overlay of chemical structures
SARS-COV-2—Severe acute respiratory syndrome coronavirus 2

1. A multi-reference poly-conformational computational method for de-novo design, optimization
and repositioning of pharmaceutical compounds as described and illustrated.
